(19) United States
(12) Patent Application Publication
(10) Pub. No.: US 2007/0173914 A1
(43) Pub. Date: Jul. 26, 2007

(54) Self-locking electrode assembly usable with an implantable medical device

(75) Inventor: Shawn D. Kollatschny, Pearland, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/338,375

(22) Filed: Jan. 24, 2006

(51) Int. Cl. A61N 1/00

(52) U.S. Cl. 607/116

(57) ABSTRACT

An electrode assembly for use with an implantable medical device. The electrode assembly comprises a spine and a plurality of electrodes that protrude from away from the spine. At least two electrodes protrude away from the spine in opposing directions.

### BACKGROUND

Implantable medical devices often stimulate body tissue by way of one or more electrodes through which the medical device electrically couples to the body tissue. For example, a vagus nerve stimulator typically includes a pulse generator that couples to the vagus nerve by way of one or more conductive leads. One or more conductive electrodes are located at or near the end of each lead. The electrodes are coupled to the nerve. Achieving sufficient direct electrical contact of the electrode with the nerve in a way that still permits the nerve to expand or bend is desirable.

### BRIEF SUMMARY

In accordance with at least one embodiment of the invention, an electrode assembly usable with an implantable medical device comprises a spine and a plurality of electrodes attached to the spine. Each electrode protrudes from the spine. At least two electrodes protrude from the spine in opposing directions and define a nerve-receiving channel. When the electrode assembly is not attached to a nerve and the electrodes are in a relaxed state position (i.e., not subjected to mechanical forces such as when the electrodes are pulled apart), the nerve-receiving channel has a cross-sectional area that is substantially less than a cross-sectional area of a nerve to which the electrode assembly is adapted to be attached. When attached to the nerve, each electrode wraps around and directly contacts at least 60% of the circumference of the nerve.

In accordance with another embodiment, an implantable medical device comprises a pulse generator, a lead assembly coupled to the pulse generator, and an electrode assembly coupled to the lead assembly. The electrode assembly comprises a spine and a plurality of electrodes attached to the spine. Each electrode protrudes from the spine, and at least two electrodes protrude from the spine in opposing directions to define a nerve-receiving channel. When the electrode assembly is not attached to a nerve and the electrodes are in a relaxed state position, the nerve-receiving channel has a cross-sectional area that is substantially less than a cross-sectional area of a nerve to which the electrode assembly is adapted to be attached. When attached to the nerve, each electrode wraps around and directly contacts at least 60% of the circumference of the nerve.

In accordance with another embodiment, an electrode assembly usable with an implantable medical device comprises a spine and a plurality of curved fingers extending from the spine. Each finger protrudes from the spine. At least two fingers protrude from the spine in opposing directions and define a nerve-receiving channel. At least one of the fingers comprises a conductor that is adapted to electrically contact a nerve. When the electrode assembly is not attached to the nerve and the fingers are in a relaxed state position, the nerve-receiving channel has a cross-sectional area that is substantially less than a cross-sectional area of a nerve to which the electrode assembly is adapted to be attached. When attached to the nerve, all of the fingers make contact with the nerve on a partial outer surface of the nerve. The partial outer surface extends circumferentially at least approximately 20% of the circumference of the nerve.

### BRIEF DESCRIPTION OF THE DRAWINGS

For a detailed description of exemplary embodiments of the invention, reference will now be made to the accompanying drawings in which:

FIG. 1 depicts, in schematic form, an implantable medical device, in accordance with a preferred embodiment of the invention, implanted within a patient and programmable by an external programming system;

FIG. 2 shows a perspective view of an electrode assembly in accordance with a preferred embodiment of the invention;

FIG. 3 shows a plan view of the electrode assembly of FIG. 2 when the electrode assembly is not attached to a nerve and is in a relaxed state;

FIG. 4 shows a perspective view of a nerve illustrating a curved portion of the circumference of the nerve that is contacted by multiple electrodes;

FIG. 5 shows multiple conductors included with the spine of the electrode assembly;

FIG. 6 shows an embodiment of the electrode assembly in which at least two electrodes adjacent one another along the spine protrude from the spine in a common direction;

FIG. 7 shows an end view of the embodiment of FIG. 6; and

FIG. 8 shows a perspective view of the electrode assembly of FIG. 6.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and is not intended to intimate that the scope of the disclosure, including the claims, is limited to that embodiment. Any numerical values provided herein are merely exemplary and do not limit the scope of this disclosure or the claims that follow, unless otherwise stated.

In the disclosure and claims that follow, the terms “couple” and “coupled” include direct and indirect electrical connections. Thus, component A couples to component B, regardless of whether component A is connected directly to component B, or connected to component B via one or more intermediate components or structures.

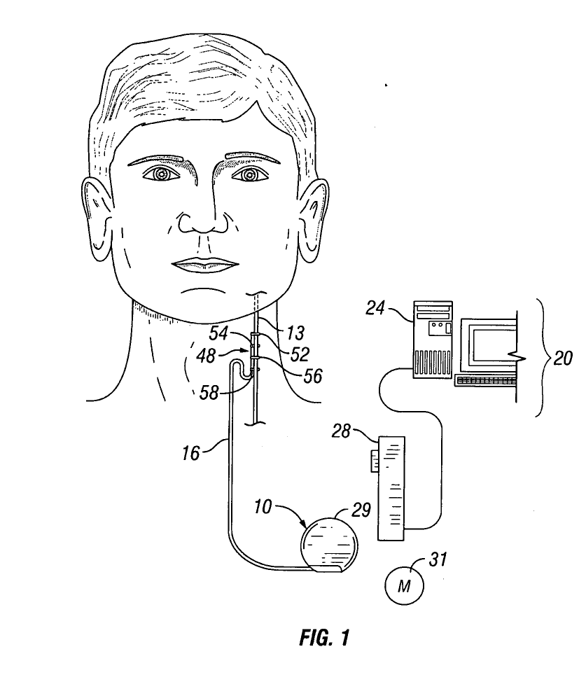

FIG. 1 illustrates an implantable medical device (“IMD”) 10 implanted in a patient. The IMD 10 may be representative of any of a variety of medical devices. At least one preferred embodiment of the IMD 10 comprises a neurostimulator for applying an electrical signal to a neural structure in a patient, particularly a cranial nerve such as a vagus nerve 13. Although the device 10 is described below in terms of vagus nerve stimulation (“VNS”), the disclosure and claims that follow, unless otherwise stated, are not limited to VNS, and may be applied to the delivery of an electrical signal to modulate the electrical activity of other cranial nerves such as the trigeminal and/or glossopharyngeal nerves, or to other neural tissue such as one or more brain structures of the patient, spinal nerves, and other spinal structures. Further still, the IMD 10 can be used to stimulate tissue other than nerves or neural tissue. An example of such other tissue comprises cardiac tissue, as in the case of implantable pacemakers and defibrillators.

Referring still to FIG. 1, a lead assembly comprising one or more leads 16 is coupled to the IMD 10 and includes one or more electrodes, such as electrodes 52, 54, 56, and 58. Each lead 16 has a proximal end that connects to a header 18 of the IMD 10 and a distal end which comprises an electrode assembly 48 containing one or more electrodes. One or more restraining members may also be provided as part of the electrode assembly to attach to the nerve and provide strain relief. The outer enclosure (or “can”) 29 of the IMD 10 preferably is, in one embodiment, electrically conductive and thus can also function as an electrode. The electrodes (which may comprise one or more of 52-58) and can 29 couple to the patient's tissue. The header 18 mates with the can 29. The header 18 contains one or more connectors to which the lead(s) 16 connect. Through conductive structures housed in the header 18, the leads electrically couple to circuitry inside the can. In at least one embodiment, the internal circuitry is implemented in the form of electrical components mounted on a printed circuit board. The electrodes, such as electrodes 52-58 and can 29, can be used to stimulate and/or sense the electrical activity of the associated tissue (e.g., the vagus nerve 13).

FIG. 1 also illustrates an external device implemented as a programming system 20 for the IMD 10. The programming system 20 comprises a processing unit coupled to a wand 28. The processing unit 24 may comprise a personal computer, personal digital assistant (PDA) device, or other suitable computing device consistent with the description contained herein. Methods and apparatus for communication between the IMD 10 and an external programming system 20 are known in the art. Representative techniques for such communication are disclosed in U.S. Pat. Nos. 5,304,206 and 5,235,980, both incorporated herein by reference. The IMD 10 includes a transceiver (e.g., a coil) that permits signals to be communicated wirelessly and noninvasively between the external wand 28 and the implanted IMD 10. Via the wand 28, the programming system 20 is capable of monitoring the performance of the IMD and downloading new programming information (e.g., data) into the device to alter its operation as desired.

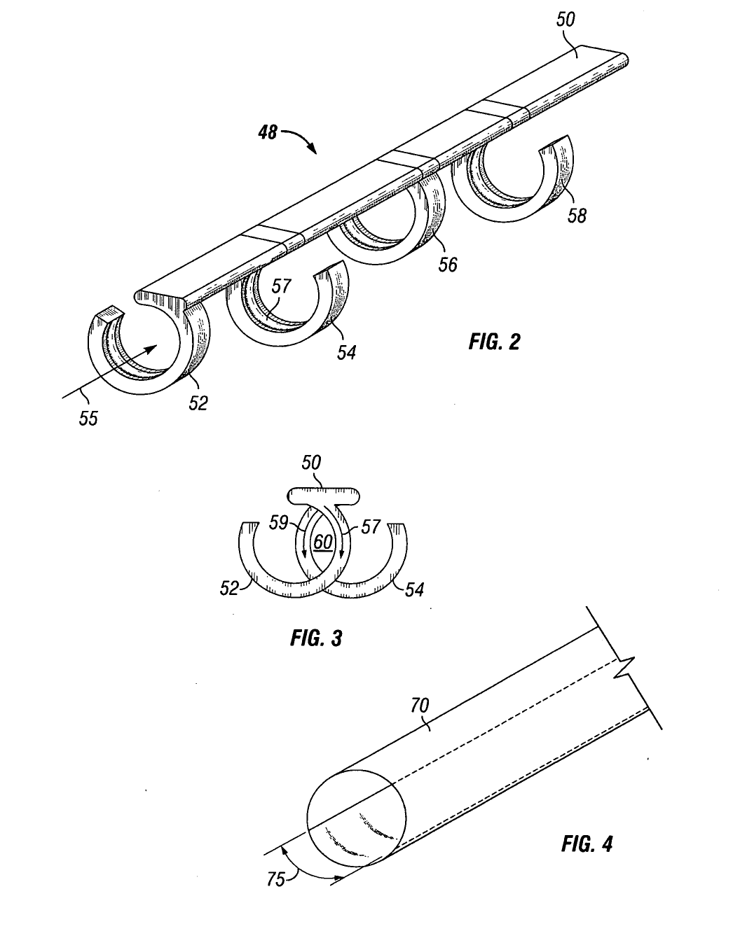

FIG. 2 shows an exemplary embodiment of an electrode assembly 48. As shown, the electrode assembly 48 comprises a spine 50 to which at least two curved fingers protrude. In the exemplary embodiment of FIG. 2, the assembly 48 comprises four curved fingers 52, 54, 56, and 58. Any one or more, or all, of the curved fingers 52-58 may comprise an electrically conductive component thereby functioning as an electrode. In some embodiments, one or more of the curved fingers are not capable of conducting electricity and, instead, function as a restraining member adapted to help hold the electrode assembly in place around the nerve.

FIG. 3 shows a plan view of the electrode assembly viewed along axis 55 (FIG. 2). The two end-most curved fingers 52 and 54 can be seen. Curved finger 52 protrudes from spine 50 in a generally clockwise direction as indicated by arrow 57. Curved finger 54 protrudes from spine 50 in a generally counter-clockwise direction as indicated by arrow 59. Any remaining curved fingers (e.g., fingers 56 and 58) generally align with curved fingers 52 and 54 and thus cannot be seen in the view depicted in FIG. 3. FIG. 3 illustrates the orientation of the fingers relative to one another when the electrode assembly is not attached to a nerve and the fingers are subjected to mechanical forces, such as when the fingers are being pulled apart to facilitate engagement with the nerve. This position is defined herein as the “relaxed” state position. As can be seen, the curved fingers protrude from the spine in opposing directions and define a nerve-receiving channel 60 therebetween. With the curved fingers in the relaxed state position, the nerve-receiving channel 60 has a cross-sectional area that is substantially less than a cross-sectional area of a nerve to which the electrode assembly 48 is adapted to be coupled. In some embodiments, the cross-sectional area of the nerve-receiving channel 60 preferably is less than approximately 80% of the cross-sectional area of the nerve. In other embodiments, the cross-sectional area of the nerve-receiving channel 60 preferably is less than approximately 60% of the cross-sectional area of the nerve.

In accordance with the preferred embodiments of the invention, the inner surface of each electrode (e.g., surface 51 that will be in contact with the nerve) is covered partially or completely with a conductor. In some embodiments, the conductor comprises an electrically conductive foil that lines at least some of the surface area of the inner surface of each electrode.

The spine 50 preferably is made from a biocompatible material such as silicone or polyurethane. The fingers 52-58 may be made from the same biocompatible material as, or different from, the spine. The spine 50 and fingers 52-58 may be formed as a unitary structure or the fingers may be made separate from, and attached to, the spine.

The material comprising each finger 52-58 preferably also is biocompatible and is elastomeric such that the finger can be deformed, at least to a certain extent, and the finger will return to its original shape and orientation upon being released from the deformed state. The fingers thus have a property referred to as “memory.” This property facilitates the fingers being spread apart so that the electrode assembly 48 can be placed on a nerve. When the fingers are released, the fingers will try to revert back to their original shape and configuration (FIG. 3). The nerve, having a cross-sectional area that is larger than the nerve-receiving channel 60 when the electrode assembly is in the relaxed state position, precludes the fingers from completely reverting back to the relaxed position. The flexibility of the fingers permits the nerve to expand and contract and remain electrically and mechanically engaged with the electrodes of the electrode assembly 48.

When the electrode assembly 48 is attached to the nerve, the fingers contact the outer surface of the nerve. Because at least two of the fingers protrude from the spine 50 in opposing directions, such fingers exert a force against the nerve generally in opposite directions. As a result, the electrode assembly “self-locks” on the nerve. Each finger wraps around and directly contacts at least a portion of the circumference of the nerve 70. In some embodiments, each finger directly contacts at least approximately 60%, and more preferably 70%, of the circumference of the nerve. Because, in such embodiments, each electrode contacts the nerve along a distance around the outer surface of the nerve that is more than half of the circumference of the nerve, an overlap region exists along the nerve as shown in FIG. 4. In particular, FIG. 4 shows a nerve 70 having a curved surface 75 extending partially around the circumference of the nerve. A plurality, and preferably all, of fingers will contact the nerve along surface 75. In some embodiments, the surface 75 extends circumferentially at least approximately 20%, and more preferably 40% of the circumference of the nerve.

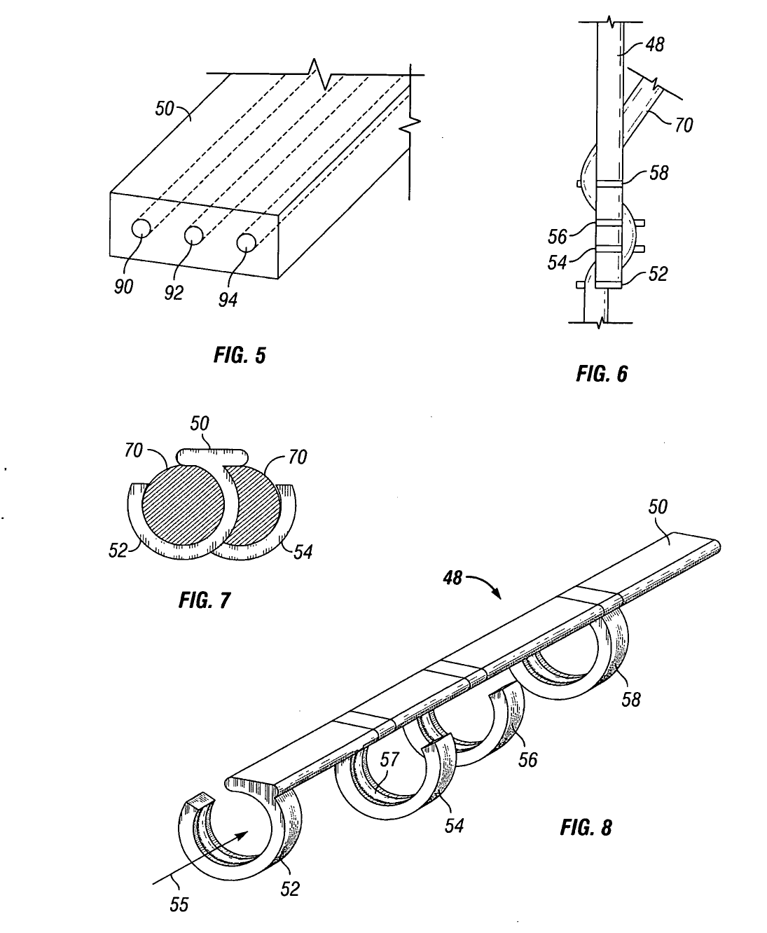

In some embodiments, all of the electrodes on the electrode assembly 48 are electrically coupled together. In other embodiments, however, two or more of the electrodes are electrically insulated and thus electrically separate from one another. FIG. 5 illustrates a cross-sectional perspective view of an embodiment of the spine 50 in which multiple conductors 90, 92, and 94 are embedded in the spine. The conductors 90-94 preferably comprise wires or other types of conductors that are embedded within the spine as the spine is formed. The spine 50 preferably is made from electrically insulative material and thus each conductor 90-94 is electrically insulated from all other conductors in the spine. Each conductor can be electrically connected to any one or more electrodes as desired. As such, the electrode assembly 48 comprises one or more electrodes and any combination of one or more electrodes can be electrically connected together.

FIG. 2 depicts an embodiment in which the fingers are arranged along the spine so that adjacent fingers protrude from the spine in opposing directions. That is, the fingers are arranged in alternating clockwise/counter-clockwise protruding directions. In other embodiments, the fingers can be configured in different arrangements. FIG. 6, for example, illustrates an embodiment in which adjacent fingers 54 and 56 protrude from the spine in the same direction, while the outer two fingers 52 and 58 protrude from the spine in the opposite direction. FIG. 7 shows an end view of the electrode assembly 48 of FIG. 6 attached to nerve 70. As can be seen in FIGS. 6 and 7, the nerve is permitted to bend while engaged with the electrode assembly 48. FIG. 8 shows a perspective view of the electrode assembly 48 of FIG. 6 showing that curved fingers 54 and 56 protrude from the spine in the same direction, which is opposite to the direction of curved fingers 52 and 58.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. An electrode assembly for an implantable medical device, comprising:
a spine; and
a plurality of electrodes protruding from the spine, wherein at least two electrodes protrude from the spine in opposing directions and define a nerve-receiving channel;
wherein, when said electrode assembly is not coupled to a nerve and said electrodes are in a relaxed state position, said nerve receiving channel comprises a cross-sectional area that is substantially less than a cross-sectional area of a nerve to which the electrode assembly is adapted to be coupled; and
wherein, when coupled to said nerve, each electrode wraps around and directly contacts at least 60% of the circumference of the nerve.

2. The electrode assembly of claim 1 wherein, when said electrode assembly is not coupled to a nerve and said electrodes are in a relaxed state position, the cross-sectional area of the nerve receiving channel is less than 80% of the cross-sectional area of the nerve.

3. The electrode assembly of claim 2 wherein, when said electrode assembly is not coupled to a nerve and said electrodes are in a relaxed state position, the cross-sectional area of the nerve receiving channel is less than 60% of the cross-sectional area of the nerve.

4. The electrode assembly of claim 1 wherein said plurality of electrodes comprises at least three electrodes and at least two electrodes adjacent one another along the spine protrude from the spine in a common direction.

5. The electrode assembly of claim 1 wherein said spine includes a plurality of electrical conductors, and wherein each conductor is coupled to at least one electrode, and is electrically insulated from all other of said electrical conductors.

6. An implantable medical device, comprising:
a pulse generator;
a lead assembly coupled to said pulse generator; and
an electrode assembly coupled to said lead assembly, wherein the electrode assembly comprises a spine and a plurality of electrodes protruding from the spine, and wherein at least two electrodes protrude from the spine in opposing directions and define a nerve-receiving channel;
wherein, when said electrode assembly is not coupled to a nerve and said electrodes are in a relaxed state position, said nerve receiving channel comprises a cross-sectional area that is substantially less than a cross-sectional area of a nerve to which the electrode assembly is adapted to be coupled; and
wherein, when coupled to said nerve, each electrode wraps around and directly contacts at least 70% of the circumference of the nerve.

7. The implantable medical device of claim 6 wherein, when said electrode assembly is not coupled to a nerve and said electrodes are in a relaxed state position, the cross-sectional area of the nerve receiving channel is less than 80% of the cross-sectional area of the nerve.

8. The implantable medical device of claim 7 wherein, when said electrode assembly is not coupled to a nerve and said electrodes are in a relaxed state position, the cross-sectional area of the nerve receiving channel is less than 60% of the cross-sectional area of the nerve.

9. The implantable medical device of claim 6 wherein said plurality of electrodes comprises at least three electrodes and at least two electrodes adjacent one another along the spine protrude from the spine in a common direction.

10. The implantable medical device of claim 6 wherein said spine includes a plurality of electrical conductors, and wherein each conductor is coupled to at least one electrode, and is electrically insulated from all other of said electrical conductors.

11. An electrode assembly usable with an implantable medical device, comprising:
a spine; and
a plurality of curved fingers protruding from said spine, wherein at least two fingers protrude from the spine in opposing directions and define a nerve-receiving channel, and wherein at least one of said fingers comprises an electrode that is adapted to electrically contact a nerve;
wherein, when said electrode assembly is not coupled to the nerve and said fingers are in a relaxed state position, said nerve-receiving channel comprises a cross-sectional area that is substantially less than a cross-sectional area of a nerve to which the electrode assembly is adapted to be coupled; and
wherein, when coupled to said nerve, all of said fingers contact the nerve on a partial outer surface of the nerve, said partial outer surface extending circumferentially at least approximately 40% of the circumference of the nerve.

12. The electrode assembly of claim 11 wherein each finger wraps around and directly contacts at least 70% of the circumference of the nerve.

13. The electrode assembly of claim 11 wherein at least two fingers comprise an electrode.

14. The electrode assembly of claim 11 wherein, when said electrode assembly is not coupled to a nerve and said fingers are in a relaxed state position, the cross-sectional area of the nerve receiving channel is less than 80% of the cross-sectional area of the nerve.

15. The electrode assembly of claim 14 wherein, when said electrode assembly is not coupled to a nerve and said fingers are in a relaxed state position, the cross-sectional area of the nerve receiving channel is less than 60% of the cross-sectional area of the nerve.

16. An electrode assembly for an implantable medical device, comprising:
a spine; and
a plurality of electrodes protruding from the spine, wherein at least two electrodes protrude from the spine in opposing directions and define a nerve-receiving channel;
wherein, when said electrode assembly is not coupled to a nerve and said electrodes are in a relaxed state position, said nerve receiving channel comprises a cross-sectional area that is less than 80% of the cross-sectional area of a nerve to which the electrode assembly is adapted to be coupled.
